(19) United States
(12) Patent Application Publication
(10) Pub. No.: US 2022/0233177 A1
(43) Pub. Date: Jul. 28, 2022

(54) SAMPLE COLLECTION SWAB

(71) Applicant: VitroDx, Inc., Fremont, CA (US)

(72) Inventor: Yunqi TIAN, Guangzhou (CN)

(21) Appl. No.: 17/585,238

(22) Filed: Jan. 26, 2022

Related U.S. Application Data
(60) Provisional application No. 63/142,463, filed on Jan. 27, 2021.

(51) Int. Cl. A61B 10/00

(52) U.S. Cl. CPC A61B 10/0051

(57) ABSTRACT

An exemplary sample collection swab comprises: a rod comprising one or more protrusions over a distal segment of the rod; and an absorbent material at least partially coating the distal segment of the rod. In some embodiments, the one or more protrusions comprise a raised helical thread.

### CROSS-REFERENCE TO RELATED APPLICATION

This application claims the benefit of U.S. Provisional Application 63/142,463 filed on Jan. 27, 2021, the entire contents of which are incorporated herein by reference for all purposes.

### FIELD OF INVENTION

The present disclosure relates generally to sample collection swabs, and more specifically to enhanced sample collection swabs with a higher absorbing capacity.

### BACKGROUND

Conventional swabs provide cone-shaped or cylindrical distal segments that have a smooth surface for collecting samples. Such a distal segment provides a limited surface area for interacting with and picking up the sample. The limited volume collected can reduce the diagnostic sensitivity and accuracy rate of the test(s) based on the collected sample, as well as increasing potential false negatives.

### BRIEF SUMMARY

Disclosed herein are enhanced sample collection swabs with a higher absorbing capacity than conventional swabs. In some embodiments, an exemplary enhanced swab comprises a screw-shaped distal segment, which offers a larger outer surface area than a conventional swab. Because the screw-shaped distal segment provides a larger surface area, it can be coated with more absorbent material and provide a larger sample collection area. In some embodiments, the fiber density can be increased. Thus, a larger volume of the sample can be collected. Further, the effective rate at which the sample is collected can be increased. In some embodiments, the conventional swab provides an absorbing capacity of approximately 70 ul, while the enhanced swab provides an absorbing capacity of approximately 270 ul.

The higher collection volume can increase the diagnostic sensitivity and accuracy rate of the test(s) based on the collected sample, as well as reducing potential false negatives. Thus, the enhanced swabs can be used for biological specimen assay and diagnostic purposes, especially for detecting patients in early stage. In particular, the enhanced swabs can be better suited for collecting virus samples (e.g., the COVID-19 virus) and can improve the diagnostic/analysis sensitivity for PCR applications.

An exemplary sample collection swab comprises: a rod comprising one or more protrusions over a distal segment of the rod; and an absorbent material at least partially coating the distal segment of the rod.

In some embodiments, the one or more protrusions comprise a raised helical thread.

In some embodiments, the one or more protrusions are arranged in a pattern.

In some embodiments, the pattern is a zigzag pattern.

In some embodiments, the rod comprises a rounded distal end.

In some embodiments, the rod is configured to be breakable at a predefined location.

In some embodiments, the rod is made of a rigid material.

In some embodiments, the rod is made of wood or plastic.

In some embodiments, the absorbent material is a fibrous material.

In some embodiments, the absorbent material is applied to the rod by electric flocking.

in some embodiments, the distal segment provides an outer surface of approximately 250 mm2.

In some embodiments, the distal segment is configured to provide an absorbing capacity of approximately 270 ul.

In some embodiments, the swab is configured to collect a virus sample.

In some embodiments, the swab is for nasopharyngeal use.

In some embodiments, the swab is for oropharyngeal use.

In some embodiments, the absorbent material is treated by plasma radiation, gamma radiation, or electric beam, any combination thereof.

In some embodiments, the absorbent material is treated to increase an absorbing capacity.

In some embodiments, the absorbent material is treated to increase a binding capacity of biological molecules.

An exemplary method of collecting a sample from an individual comprises: contacting a swab with a site in or on the individual, wherein the swab comprises: a rod comprising one or more protrusions over a distal segment of the rod and an absorbent material at least partially coating the distal segment of the rod; and collecting the sample from the site from the individual via the absorbent material.

In some embodiments, the sample comprises a virus.

In some embodiments, the virus is the COVID-19 virus or a variation thereof.

In some embodiments, the site is within the individual's head.

In some embodiments, the method comprises placing the distal segment of the rod in a container; and breaking the distal segment off from the rod at a predefined location on the rod.

### DESCRIPTION OF THE FIGURES

FIG. 1A illustrates a base rod of an exemplary conventional swab.

FIG. 1B illustrates exemplary dimensions of the base rod of the conventional swab.

FIG. 2A illustrates a base rod of an exemplary enhanced swab, in accordance with some embodiments.

FIG. 2B illustrates exemplary dimensions of the base rod of the enhanced swab, in accordance with some embodiments.

FIG. 3A illustrates conventional swabs with and without the absorbent coating, in accordance with some embodiments.

FIG. 3B illustrates enhanced swabs with and without the absorbent coating, in accordance with some embodiments.

FIG. 4 illustrates exemplary packaging, in accordance with some embodiments.

### DETAILED DESCRIPTION

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

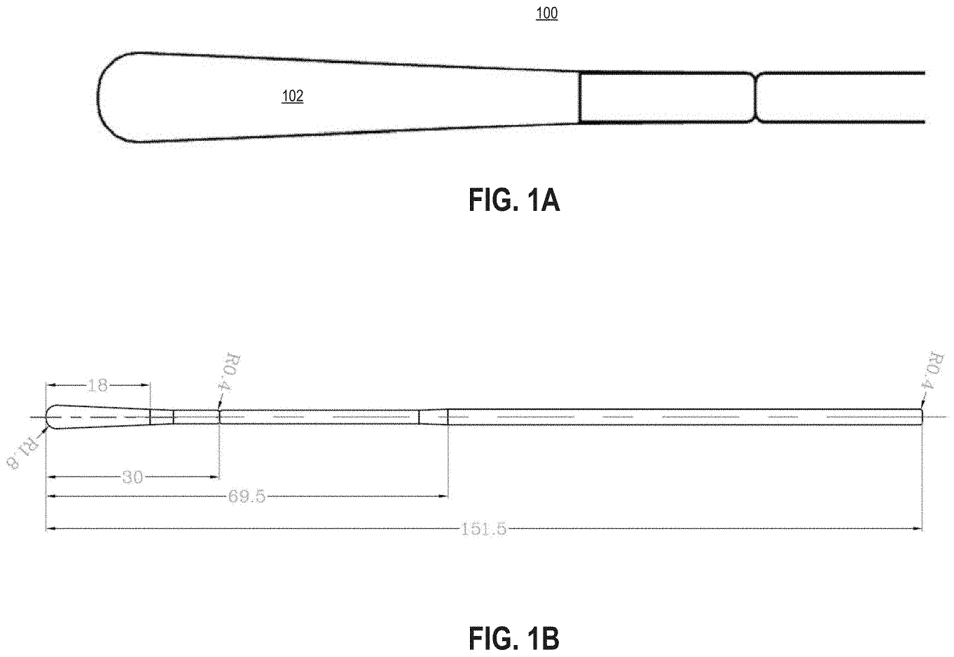

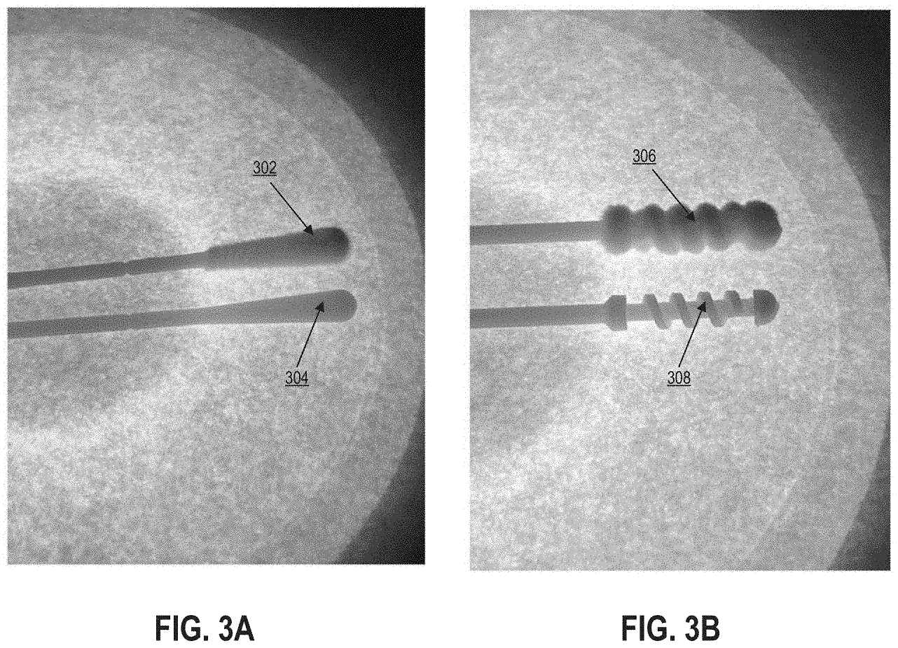

FIG. 1A illustrates a base rod 100 of a conventional sample collection swab. The conventional swab provides a smooth surface for collecting samples. In the depicted example, the rod comprises a cone-shaped distal segment 102, but the distal segment 102 can be of other shapes (e.g., cylindrical shape) to provide a smooth surface for collecting samples. The distal segment 102 can be coated with an absorbent material (not depicted), such as cotton or other fibrous materials. For example, FIG. 3A illustrates a base rod 304 and a swab 302 that comprises both a base rod and absorbent material applied around the distal segment. The rod 100 can be made of a rigid material such as plastic.

In operation, the rod 100 can be placed such that its distal segment 102 is at least partially in contact with the sample (e.g., a liquid biological specimen) to be collected. The absorbent material coating the distal segment can absorb the sample. The amount of the sample collected is at least partially dependent on the outer surface of the distal segment of the rod.

FIG. 1B illustrates exemplary dimensions in millimeters of the base rod. In particular, the base rod can be 151.5 mm in total length, and the distal cone-shaped segment can be 18 mm in length. The cross-sectional radius (“R”) of the rod at the distal end is 1.8 mm, and the cross-sectional radius of the rod at the proximal end is 0.4 mm. In the depicted example, the 18 mm cone-shaped distal segment has an outer surface area of 194.8 mm².

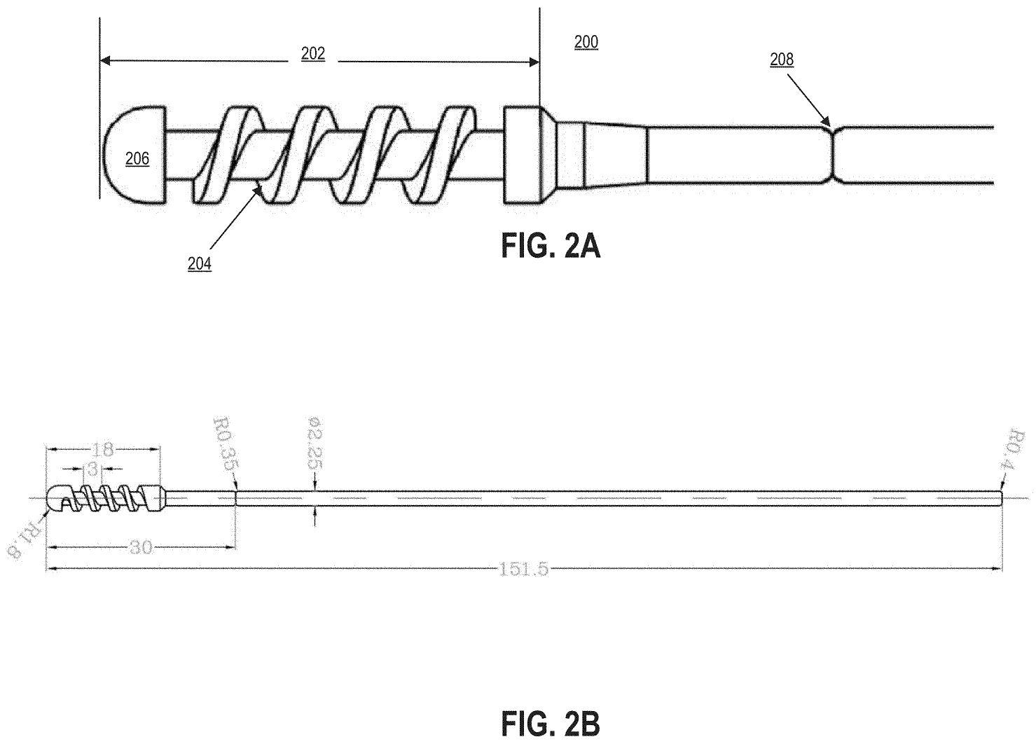

FIG. 2A illustrates a base rod 200 of an exemplary enhanced swab, in accordance with some embodiments. The rod 200 has one or more protrusions on the distal segment to provide more surface area for collecting the sample. In the depicted example, the rod 200 has a screw-shaped distal segment 202. The distal segment 202 comprises a raised helical thread 204 running around the rod. In the depicted example, the distal segment 202 further comprises a rounded distal end 206, which can prevent the rod from harming the subject when it comes into contact with the subject. The distal segment 202 can be coated with an absorbent material (not depicted), such as cotton or other fibrous materials. In some embodiments, the absorbent material can be applied as electric flocked fibers. For example, FIG. 3B illustrates a base rod 308 and an enhanced swab 306 that comprises both a base rod and a coating material applied around the distal segment. The rod 200 can be made of a rigid material such as plastic.

In operation, the rod 200 can be placed such that its distal segment 202 is at least partially in contact with the sample (e.g., a liquid biological specimen) to be collected. The absorbent material coating the distal segment can absorb the sample. The amount of the sample collected is at least partially dependent on the outer surface of the distal segment of the rod.

FIG. 2B illustrates exemplary dimensions in millimeters of the base rod 200 of the enhanced swab. Like the base rod 100, the base rod 200 can be 151.5 mm in total length, and the distal cone-shaped segment can be 18 mm in length. Also like the base rod 100, the radius (“R”) of the rod 200 at the distal end is 1.8 mm, and the radius of the rod at the proximal end is 0.4 mm.

The screw-shaped distal segment 202 comprises a larger outer surface area than the cone-shaped distal segment 102. In the depicted example, the 18 mm distal screw-shaped segment has an outer surface area of 250.2 mm², which is larger than the outer surface area of the cone-shaped distal segment. It should be appreciated that the dimensions provided in FIGS. 1B and 2B are merely exemplary to show that a conventional swab and an enhanced swab of identical length and distal radius can provide different surface areas and thus have different absorbing capacities. The enhanced swabs can have other dimensions without departing from the spirit of the invention.

Because the screw-shaped distal segment 202 provides a larger surface area, it can be coated with a larger amount of absorbent material and provide a larger sample collection area. In some embodiments, the fiber density can be increased. Thus, a larger volume of the sample can be collected. Further, the effective rate at which the sample is collected can be increased. in some embodiments, the conventional swab provides an absorbing capacity of approximately 70 ul, while the enhanced swab provides an absorbing capacity of approximately 270 ul.

The larger collection volume can increase the diagnostic sensitivity and accuracy rate of test(s) based on the collected sample, as well as reducing potential false negatives. Thus, the enhanced swabs can be used for biological specimen assay and diagnostic purposes, especially for detecting patients in early stage. In particular, the enhanced swabs can be better suited for collecting virus samples (e.g., the COVID-19 virus) and can improve the diagnostic/analysis sensitivity for PCR applications.

it should be appreciated that the distal segment 202 of the enhanced sample collection swab can have other shapes. Other types of protrusions (e.g., arranged in a different pattern) can be applied at the distal segment to increase the outer surface of the distal segment of the rod. In some embodiments, the distal segment comprises one or more protrusions that are arranged in a zigzag pattern.

In some embodiments, the enhanced swab can be modified/treated by plasma radiation, gamma radiation, and/or electric beam to further improve its absorbency for specimen collection. The improved absorbency can increase the amount of biological specimen collected by each swab and thus improve its diagnostic value. For example, the absorbent material on the swab can be treated before and/or after it has been applied to the rod. In a first exemplary experiment, an enhanced swab that is not treated by plasma or gamma radiation has an absorbing capacity of about 100 μl, an enhanced swab that is treated by plasma radiation has an absorbing capacity of about 140 μl (i.e., an increased 40% capacity); an enhanced swab that is treated by gamma radiation has an absorbing capacity of about 130 μl (i.e., an increased 30% capacity). In a second exemplary experiment, an enhanced swab that is not treated by plasma or gamma radiation has an absorbing capacity of about 100 μl; an enhanced swab that is treated by plasma radiation has an absorbing capacity of about 140 μl (i.e., an increased 40% capacity); an enhanced swab that is treated by gamma radiation has an absorbing capacity of about 125 μl (i.e., an increased 25% capacity).

In some embodiments, the enhanced swab can be modified/treated to provide a hydrophilic surface to further improve its binding capacity of biological molecules (such as proteins and nucleic acids) for specimen collection. The improved binding capacity can increase the amount of biological specimen collected by each swab and thus improve the swab's diagnostic value. The absorbent material on the swab can be treated before and/or after it has been applied to the rod. In certain embodiments, the absorbent material on the swab can be treated by oxygen-hydrogen plasma radiation to add ionized groups (such as —COOH, —OH, etc.) for hydrophilicity, which can increase the binding capacity to approximately 98%. In other embodiments, the absorbent material on the swab can be treated by gamma radiation to break chemical structures of the absorbent material (e.g., nylon fibers and polystyrene (PS)). In some embodiments, the absorbent material on the swab can be treated by electric beam.

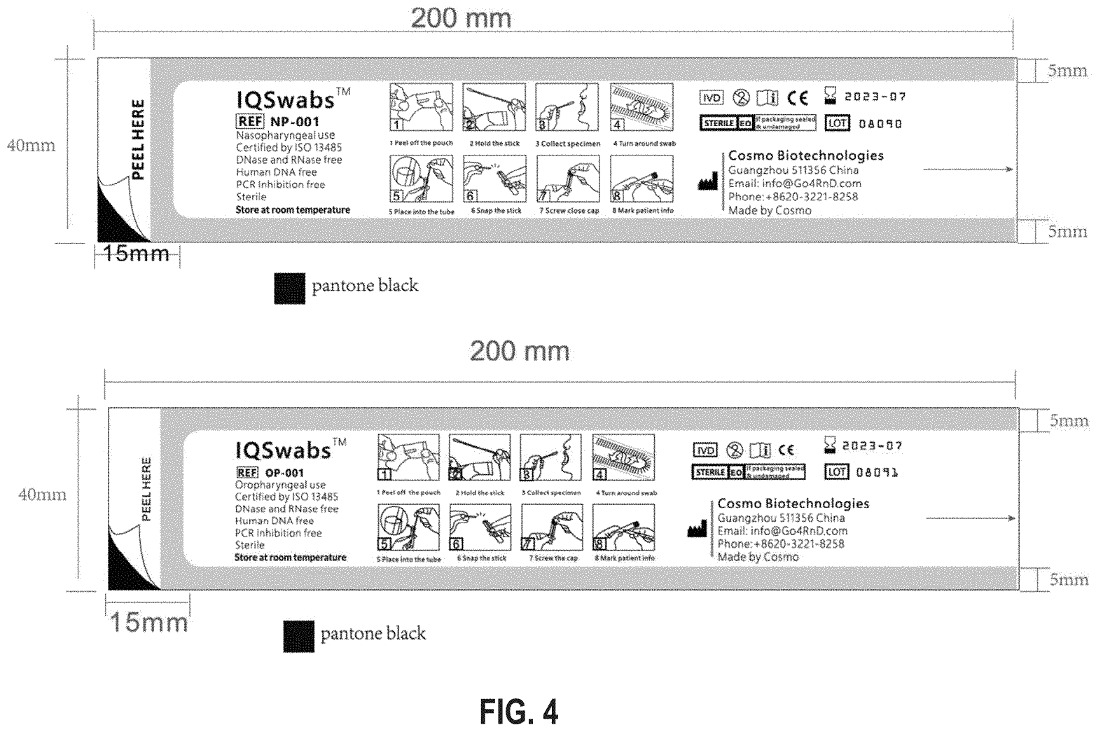

FIG. 4 illustrates exemplary packaging for a sample collection swab, in accordance with some embodiments. In the depicted examples, the packaging can indicate that swab is designed for nasopharyngeal use and/or oropharyngeal use. As shown, the packaging can provide instructions for using the swab to collect a sample, which can include 1) peel off the pouch, 2) hold the stick, 3) collect specimen, 4) turn around swab, 5) place into the tube, 6) snap the stick, 7) screw close cap, and 8) mark patient information. In particular, the swab can be snapped at, for example, a predefined location 208 (FIG. 2A).

In some embodiments, an exemplary method collecting a sample from an individual comprises: contacting a swab (e.g., an enhanced swab with a screw-shaped distal segment 202) with a site in or on the individual, and collecting the sample from the site from the individual via the absorbent material coating the distal segment of the swab. In some embodiments, the sample comprises a virus (e.g., the COVID-19 virus or a variation thereof). In some embodiments, the site is within the individual's head (e.g., into the cavity between the nose and mouth). For example, testing for COVID-19 involves inserting the enhanced swab into the cavity between the nose and mouth (nasopharyngeal swab) over a predefined period of time and rotating it several times.

In some embodiments, the method further comprises placing the distal segment of the rod in a container (e.g., a tube) and breaking the distal segment off from the rod at a predefined location on the rod (e.g., 208 in FIG. 2A), The container can be then sealed, labeled with the individual's information, and transported to a testing location where the collected sample is analyzed.

Although the disclosure and examples have been fully described with reference to the accompanying figures, it is to he noted that various changes and modifications will become apparent to those skilled in the art. Such changes and modifications are to be understood as being included within the scope of the disclosure and examples as defined by the claims.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

What is claimed is:

1. A sample collection swab, comprising:
a rod comprising one or more protrusions over a distal segment of the rod; and
an absorbent material at least partially coating the distal segment of the rod.

2. The swab of claim 1, wherein the one or more protrusions comprise a raised helical thread.

3. The swab of claim 1, wherein the one or more protrusions are arranged in a pattern.

4. The swab of claim 3, wherein the pattern is a zigzag pattern.

5. The swab of claim 1, wherein the rod comprises a rounded distal end.

6. The swab of claim 1, wherein the rod is configured to be breakable at a predefined location.

7. The swab of claim 1, wherein the rod is made of a rigid material.

8. The swab of claim 1, wherein the rod is made of wood or plastic.

9. The swab of claim 1, wherein the absorbent material is a fibrous material.

10. The swab of claim 1, wherein the absorbent material is applied to the rod by electric flocking.

11. The swab of claim 1, wherein the distal segment provides an outer surface of approximately 250 mm².

12. The swab of claim 1, wherein the distal segment is configured to provide an absorbing capacity of approximately 270 ul.

13. The swab of claim 1, wherein the swab is configured to collect a virus sample.

14. The swab of claim 1, wherein the absorbent material is treated by plasma radiation, gamma radiation, or electric beam, any combination thereof.

15. The swab of claim 1, wherein the absorbent material is treated to increase an absorbing capacity.

16. The swab of claim 1, wherein the absorbent material is treated to increase a binding capacity of biological molecules.

17. The swab of claim 1, wherein the swab is for nasopharyngeal use.

18. The swab of claim 1, wherein the swab is for oropharyngeal use.

19. A method of collecting a sample from an individual, comprising:
contacting a swab with a site in or on the individual, wherein the swab comprises:
a rod comprising one or more protrusions over a distal segment of the rod and an absorbent material at least partially coating the distal segment of the rod; and
collecting the sample from the site from the individual via the absorbent material.

20. The method of claim 19, wherein the sample comprises a virus.

21. The method of claim 19, wherein the virus is the COVID-19 virus or a variation thereof.

22. The method of claim 19, wherein the site is within the individual's head.

23. The method of claim 19, further comprising:
placing the distal segment of the rod in a container; and
breaking the distal segment off from the rod at a predefined location on the rod.
